(19) United States
(12) Patent Application Publication
(10) Pub. No.: US 2022/0057401 A1
(43) Pub. Date: Feb. 24, 2022

(54) SET OF REAGENTS FOR DETECTING A MARKER OF EPITHELIAL CARCINOMAS

(71) Applicant: JVS DIAGNOSTICS LIMITED LIABILITY COMPANY, Moscow (RU)

(72) Inventors: Zhanneta Rashidovna CHERKASOVA, D. Saburovo (RU); Sergei Alexandrovich TSURKAN, Moscow (RU); Viatcheslav Borisovich KONDRATIEV, Moscow (RU)

(21) Appl. No.: 16/972,895

(22) PCT Filed: May 28, 2019
(86) PCT No.: PCT/RU2019/050069

§ 371 Date: Nov. 3, 2021

(30) Foreign Application Priority Data
Jun. 7, 2018 (RU) 2018121107

(51) Int. Cl. G01N 33/574

(52) U.S. Cl. CPC G01N 33/57469; G01N 2333/46

(57) ABSTRACT

The invention relates to the new application of medicine, in particular to the laboratory cancer diagnostics. It describes a set of reagents for detecting the marker for epithelial carcinomas CA-62 in patients' blood serum for early detection of cancer, detecting cancer recurrences and monitoring cancer treatment of human epithelial tumors using immunochemical analysis. The main difference between the CA-62 cancer marker and other well-known markers comprises of the fact that the maximum peak of its expression falls on the very beginning of cancer development. This makes it possible to detect the early stages of tumor growth with high sensitivity and specificity when the cure rate is at maximum. The dynamics of the CA-62 marker level also show high efficacy in detecting tumor recurrences and monitoring the treatment of patients with advanced cancer. The set of reagents includes a microplate with immobilized antibodies to a CA-62 cancer antigen, CA-62-Acridinium conjugate, CA-62 standard calibrators in the measurement range from 10 to 30000 U/ml, CA-62 positive control, working buffer solution (pH 2.0-8.0) containing Tween-80.

### A BRIEF DESCRIPTION OF THE INVENTION

Early stages of cancer are characterized by a lack of any clinical indication of a disease development. This leads to the delay in diagnosis of cancer and results in poor clinical outcomes and increased mortality risk in patients [1]. One of the suitable methods for early cancer detection is the identification of the specific biomolecules, “cancer markers” that were found in serum, tissues or fluids of the patients, which are developed during cancer development. Most of currently known tumor markers are tissue-specific; however, they do not have high sensitivity for early stages of cancer [2-5].

As compare to CEA, PSA, CA-125 and other cancer markers, the novel marker for epithelial carcinomas, N-glycoprotein CA-62, is a highly sensitive tumor-associated protein aimed precisely for the detection of very early stages of epithelial carcinomas, in particular, adenocarcinomas of the prostate, breast, and ovaries. The unique difference between novel CA-62 cancer marker and other well-known markers comprises of the fact that the maximum peak of its expression falls on the very beginning of cancer development as compared to the later stages of the disease, when the cancer has metastasized. Therefore, the measurement of the serum level of the CA-62 antigen allows the identification of the early stages of cancer, when five-year survival rate is high. To date, cohorts of serum samples from patients with the early stages of prostate cancer, breast cancer, and ovarian cancer have been studied using CA-62 biomarker. These types of cancer demonstrate a high level of expression of the CA-62 cancer marker, which makes it possible to use in combination with other organ-specific tumor markers such as PSA, CA-125, CEA or CA 15-3, to increase the sensitivity of the latter when detecting the earliest stages of the prostate, breast and ovarian cancers. Moreover, the CA-62 cancer marker can be an accurate tool for cancer verification or for conducting a differential diagnosis between benign and malignant tumors. Furthermore, the dynamics of the CA-62 marker's level shows high efficacy in detecting cancer recurrence and in monitoring cancer treatment of patients with advanced cancer. The studies are being prepared for publication.

Screening programs for detecting early stages of cancer by measuring serum level of cancer markers such as PSA, CEA, CA-125, and CA 15-3 as indicators for more in-depth diagnosis of these patients are broadly implemented in Western Europe and Canada. However, none of the officially recognized cancer markers have a perfect combination of a 100% specificity and a 100% sensitivity and results in false negative results for early stages of cancer [5-7]. Furthermore, benign tumors in patients produce a lot of false positive results, which substantially lead to overdiagnosis of cancer, especially cancer of the prostate gland [8-10]. It leads to an increased number of biopsies in patients with benign prostate hyperplasia. However, it is an invasive diagnostic procedure that is associated with approximately 30% of severe complications.

### DETAILED DESCRIPTION OF THE INVENTION

A novel diagnostic test for the detection of the marker for epithelial carcinomas allows identification of the novel biomarker, cancer-associated N-glycoprotein CA-62 from the surface of epithelial cancer cells in the patients' serum. Furthermore, this test specifically detects the very beginning of oncogenesis. Epithelial cancer cells secrete a soluble fraction of N-glycoprotein into the blood, which allows quantitative measuring of CA-62 marker and makes it possible to use as a diagnostic method for early detection of epithelial carcinomas, screening of patients at risk and monitoring the treatment of epithelial cancers. Measurement of serum CA-62 level can be carried out along with physical observations and instrumental diagnostic methods such as an ultrasound, X-ray, and MRI.

N-glycoprotein with a M.W. of about 62 kDa, consisting of an N-glycosylic Asn-GlcNAc bond and several external highly branched fucosylated polysaccharide antennas that are formed in large quantities on the surface of malignant epithelial cells from the beginning of cancer formation, is a specific marker for epithelial carcinomas. Epithelial tumors of the prostate gland, mammary gland or ovary are developed from the superficial or glandular epithelium. They are distinguished by the appearance of atypical epithelial cells, their high proliferation rate, which can progress and transform into squamous cell carcinoma. The process begins in the transitional epithelium cells, which are a squamous metaplastic epithelium of various degree of maturity that is not fully differentiated. The metaplastic epithelium is considered to be the source for the transitional zone, the most vulnerable site for external influences. In the presence of immature epithelium neoplasia may develop within the transitional zone.

Specific monoclonal antibodies (MaT-anti-CA-62) previously developed by our team recognise a tumor-specific oligosaccharide fragment on the surface of the transformed epithelial cancer cells. It allows identifying all the molecular forms of the glycoprotein CA-62. A novel diagnostic set of reagents was developed based on the previously identified unique monoclonal antibodies against N-glycoprotein CA-62. It is used to measure the CA-62 tumor marker's level in serum and to help with the detection of the early stages of malignant epithelial carcinomas in patients, in particular, adenocarcinoma of the prostate gland, adenocarcinoma of the mammary gland or ovarian carcinoma.

Another important problem is monitoring treatment response of cancer patients during therapy. It is necessary to evaluate the treatment efficiency and to make timely decisions in regards to any changes to the chemotherapy treatment plan. Currently, for monitoring the progression of the disease are mainly used expensive instrumental methods of analysis since there are no sensitive biomarkers that can correctly reflect the tumor response to the treatment. A pilot clinical study conducted by us at the N.N. Blokhin showed that the CA-62 tumor marker can be effectively used to monitor chemotherapy in patients with stage III-IV small cell lung cancer (SCLC), gastric cancer (GC) and colorectal cancer (CRC).). The dynamics of CA-62 marker's changes during chemotherapy in patients with gastric cancer, SCLC and CRC showed a good correlation (79%) of the CA-62 marker with the general condition of patients in accordance with the RECIST criteria.

The recurrence and metastasis of a malignant tumor is a serious complication that is more dangerous for the patient's life than the primary tumor. Early detection of these complications and a timely treatment are the main approaches to increase the life expectancy of cancer patients. Our study on patients with advanced cancers showed that the dynamics of the CA-62 marker could be a particularly accurate tool for detecting tumor recurrences.

Serum markers of the mucin glycoprotein family are currently the most frequently used tool for the diagnosis of breast cancer. CA 15-3 marker, a member of this family, is the most sensitive and specific glycoprotein. A positive reaction to the CA 15-3 marker is observed in approximately 10% of patients with stage I, in 25% with stage II, and about 45% in stage III [11]. Therefore, in addition to CA 15-3, another most frequently used marker for the diagnosis of malignant tumors of the breast is the cancer-embryonic antigen CEA [12]. Thus, due to the low sensitivity of currently known biomarkers, it is possible to reliably detect the early stages of breast cancer only with the mammography. The results of CA 15-3 and CEA cancer markers are the most informative for the recognition of relapses and metastases. The combination of these markers makes it possible to detect early metastases in the bone and liver at least in 60% of cases.

Prostate-specific antigen (PSA) is used to diagnose prostate cancer and formed in large quantities by epithelial cells [7-9]. The PSA level in healthy men ranges from 0 to 4 ng/ml, and an increased level is usually associated with prostate diseases. Therefore, this marker is widely used to diagnose and monitor the treatment of the prostate cancer. Advantages of the PSA method: 1. High specificity of the test up to 93.6% with the differentiation between healthy individuals and cancer patients practically does not give false positive results for healthy men. 2. In some cases (20-30%) it allows to detect prostate cancer in the early stages, when the chances of cure are at maximum. Disadvantages of the method: 1. High percentage of false-positive results that do not allow distinguishing malignant prostate diseases from other benign diseases of the prostate such as prostatic hyperplasia. This leads to unnecessary biopsies of the prostate gland. 2. Low sensitivity of the test (about 30%) when detecting early stages of cancer of the prostate. 3. In about 1 out of 4 cases, the PSA test cannot detect prostate cancer when used without combination with other tests, with 30% of men with cancer remaining undetected [9].

Cancer antigen CA-125 is currently used for diagnosis, disease progression and response to therapy. Despite the fact that CA-125 is a well-established biomarker for ovarian cancer, it has low sensitivity in determining early stages and it has low specificity in differentiating malignant from benign diseases [13-14]. CA-125 is a glycoprotein antigen 125, mucin-16 that is used as ovarian cancer biomarker and its metastases. Reference values for CA-125 biomarker in laboratory diagnostics: less than 35 U/ml for healthy women. Advantages of the method: 1. A sufficiently high sensitivity of the test (up to 80%) in detecting the late stages of ovarian cancer. In most cases of ovarian cancer, CA-125 levels rise more than 5 times. 2. The test has a good prognostic ability in monitoring the treatment of ovarian cancer, diagnosing its metastasis and evaluating the effectiveness of therapy. Disadvantages of the method: 1) Low specificity of CA-125 biomarker (from 60% and below) in the differentiation between malignant and benign diseases of the female reproductive system; 2) CA-125 test cannot be used for early diagnosis of ovarian cancer due to its low sensitivity (less than 60%), since in most patients with early stage of ovarian cancer, the level of CA-125 cancer antigen is within the normal range below 35 U/ml.

Based on our highly specific anti-CA-62 monoclonal antibodies, we developed a diagnostic set of reagents for detecting the marker for epithelial carcinomas CA-62 in patients' serum. This set of reagents has a unique cancer-specificity for epithelial tumors, which helps to detect with a high probability the presence of different carcinomas localizations at the early stages, timely identify tumor recurrences and effectively monitor the treatment of cancer patients. The scope of the set is clinical laboratory diagnostics, clinical medicine (oncology).

The set of reagents includes:

1. A microplate with immobilized specific mouse monoclonal antibodies to CA-62 cancer antigen that is ready for use and does not require additional preparation. It must be kept frozen and stored at −20° C. for the entire shelf life of the set of reagents. Mouse IgM monoclonal antibodies against the CA-62 glycoprotein antigen (62 kDa) were isolated from own hybridoma cell line. Mouse monoclonal antibodies against the marker for epithelial carcinomas CA-62 were purified by affinity chromatography following the developed laboratory operation protocols. Binding specificity of the isolated monoclonal antibodies to the CA-62 cancer antigen was confirmed using PAGE electrophoresis, Western blotting with the labelled antigen, as well as using chemiluminescence immunoassay (CLIA): direct binding of the antibody to the antigen, as well as competent analysis with the native CA-62 antigen.
2. The CA-62-Acridinium conjugate is a concentrated solution of a conjugate with a luminescent label, packaged in 1.5 ml vial, 100 μl each with a minimum concentration of 300 μg/ml in 100×10⁻³ M PBS buffer (pH 7.2). A conjugate CA-62-Acridine contains 0.02% of Na Merthiolate, a protein stabilizer. The conjugate should be frozen and stored at −20° C. for the entire shelf life of the set of reagents.
3. Standard calibrators of CA-62 in the range from 10 to 30,000 U/ml; 500 μl; 1 ml vial. They represent a standard panel of biomarker calibrators, which are calibration probes with different concentrations of the CA-62 cancer antigen, in the indicated concentration range, packaged in 1 ml vials of 500±20 μl each. The standard calibrators contain the indicated amount of CA-62 cancer antigen in the working buffer solution (pH 2.0-8.0), containing Tween-80, 0.02% Na Merthiolate, and does not require further dilution. Standard calibrators should be kept frozen and stored at −20° C. for the entire shelf life of the set of reagents.
4. The CA-62 positive control, 250 μl; 1 ml vial. It has a predetermined concentration of a cancer antigen in the range from 10 to 30000 U/ml. It is designed to control the suitability of the set of reagents during its storage, and it is not mandatory for use during the test. The CA-62 positive control is packaged in 1 ml vials of 250±10 μl. The sample contains the amount of CA-62 cancer antigen specified in the label in U/ml in the working buffer solution (pH 2.0-8.0), containing Tween-80, 0.02% Na Merthiolate, and does not require further dilution. The positive control CA-62 should be kept frozen and stored at −20° C. for the entire shelf life of the set of reagents.
5. Working buffer solution (pH 2.0-8.0), containing Tween-80, packaged in a 25 ml vial, frozen, does not require further dilution. The working buffer solution contains 6 ppm (6.5×10⁻³ M) of the antimicrobial drug Bioneutral C5. When defrosted, the buffer solution should be clear, with a yellow colour and slight foaming, caused by the presence of Tween-80. The buffer solution should be kept frozen at a temperature of −20° C. for the entire shelf life of the set of reagents.

The set of reagents of this invention has an increased sensitivity (from 80 to 95%) and specificity (from 80 to 95%) depending on the type of cancer. This invention can be used in medical diagnostic laboratories.

### Principle of the Method

The set of reagents “Marker for epithelial carcinomas CA-62” was developed based on a serum test, which consists of a single-step competitive solid-phase chemiluminescence immunoassay (CLIA) using highly specific monoclonal antibodies to CA-62 glycoprotein. The immunoassay based on the direct competition between the labelled CA-62 antigen and the native cancer antigen contained in the sample for binding to specific anti-CA-62 monoclonal antibodies immobilized in the wells.

During 2-hours incubation process, a solution containing a diluted serum sample and a fixed concentration of CA-62 labelled with a luminescent label is added to the antibodies immobilised in the wells of the microplate. The sorbent is then washed from unbound components, and the luminescent signal formed on the solid phase of the immune complexes is recorded {Antibody-(CA-62-Acridinium)}.

The magnitude of the recorded luminescent signal is inversely proportional to the concentration of the CA-62 antigen in the sample. A series of standard calibrators is tested simultaneously with the samples. A calibration curve (Logit-Log curve) is then generated for the quantitative determination of the CA-62 epithelial carcinoma marker in units of measurement (U/ml) in unknown samples.

### The Procedure for Applying the Set of Reagents Includes the Following Stages:

### Stage 1. Reagents Preparation

Before conducting the test, it is necessary to open the package of the set of reagents, remove all components, including the sealed bag with the immobilized microplate. All the components of the set of reagents and the samples to be analysed should be incubated at room temperature (22-25° C.) for at least one hour. For partial use of the set, after sampling a part of the contents, the vials and the remaining stripes of the microplate should be immediately tightly closed, placed in a refrigerator and stored at a temperature from −20 to −30° C. within the shelf life of the set of reagents.

Standard calibrators, a positive control, a microplate with the immobilized antibodies and a working buffer solution (pH 2.0-8.0), containing Tween-80, are ready for use and do not require additional dilution. Test system “CA-62” conjugate requires dilution to obtain a working solution using a buffer solution. The exact dilution of the CA-62-Acridinium conjugate is indicated on the label of each batch and in the passport of the reagent set series. A diluted solution of the conjugate must be used within an hour to avoid loss of the luminescent signal. It is recommended to dilute the working solution of the conjugate in a centrifuge tube with a screw cap such as Falcon and to wrap the tube with aluminium foil.

Test tubes with standard calibrators CA-62 should be gently mixed on a vortex to avoid any foam formation during pipetting. It is important to change the tip after pipetting of each Calibrator. If required, the Positive Control CA-62 can be used in the test, which is not mandatory during the test and is intended only to control the suitability of the set of reagents during storage. The Positive Control CA-62 is applied to the wells of the microplate in a volume of 50 μl/well. The Positive Control CA-62 should be stored frozen at −20° C. for the entire shelf life of the set of reagents.

1. Microplate Preparation

The package should be opened, and the required number of strips should be placed on the frame for the analysis. A microplate with immobilized antibodies is ready for use. Unused strips should be immediately placed into a package with a desiccant. The air should be removed, and the package should be tightly closed. It should be placed in a freezer and stored at a temperature from −20° C. to −30° C. for the entire shelf life of the set of reagents.

2. Preparation of Working Buffer Solution

A working buffer solution (pH 2.0-8.0) containing Tween-80, is ready for use and does not require further dilution. It is necessary to defrost the vial with the working buffer solution, bring it to room temperature and pour the required amount of solution into the bath for buffer solutions and a Falcon tube. Working buffer solution contains a high concentration of Tween-80, which causes the foaming of the solution within the normal range. Dispose of the remaining buffer solution after use. It is not allowed to drain the rest of the buffer solution into the vial with the initial solution.

3. Preparation of CA-62-Acridinium Conjugate

The CA-62-Acridine conjugate is a concentrated solution of cancer antigen labelled with luminescent Acridinium in 100×10⁻³ M phosphate-saline buffer solution at a concentration of 300 μg/ml. It must be diluted to the concentration indicated on the label of the conjugate (or indicated in the passport series) to obtain a diluted solution of the conjugate.

4. Preparation of the Samples to be Analysed and their Storage

For the correct implementation of the CA-62 test system, all the samples to be analysed must undergo preliminary preparation. It is involves a heat treatment of all the samples in a water bath at 56° C. for 30 minutes (with sample volumes from 0.5 ml to 1 ml). Failure to follow this procedure will change the test results. Samples with preservative may be stored for 3 weeks at 4° C. until use. For longer storage, the samples should be frozen at a temperature from −20° C. to −70° C.

Dilution of the analysed samples 1:10 is made in the wells of the microplate by mixing 10 μl of the sample with 40 μl of working buffer solution. Following this step, 50 μl of the diluted solution of CA-62-Acridine conjugate is then added to the mixture.

### Stage 2. Conducting CLIA “Set of Reagents for Detecting the Marker of Epithelial Carcinomas”

All the reagents, standard calibrators, and samples should be brought to room temperature before performing the test and well mixed before use. Standard calibrators and test specimens are processed uniformly and should be tested in duplicate. Diluted standard calibrator solutions, diluted samples and the working solution of CA-62-Acridine conjugate should be freshly prepared. Do not use pre-diluted and stored materials, as this may alter the measurement results of the test.
1. Use the required number of strips from the microplate with immobilized antibodies to CA-62 to perform the analysis.
2. Add 50 μl of standard calibrators CA-62 into wells A-H in duplicate.
3. Using a single-channel pipette with a variable volume, add 10 μl of each sample in two adjacent empty wells (in duplicate) with immobilised antibodies. Place a drop of serum on the left side of the bottom of the microplate, while changing the tip each time. The time required for adding samples should not exceed 30 minutes when using all wells.
4. Using an 8-channel pipette, add 40 μl of working buffer solution with Tween-80 to the wells for intermediate dilution of the samples: this will prevent them from drying out.
5. Thoroughly mix the samples in the wells by gradually patting different sides of the microplate.
6. Dilute the CA-62-Acridinium conjugate using an indicated on the bottle dilution factor to obtain a diluted solution of the conjugate with a working concentration of X μg/ml. For all the wells, 6 ml of a diluted solution of the conjugate in a working buffer (pH 2.0-8.0), containing Tween-80, should be prepared. Gently mix the resulting diluted conjugate solution with a vortex, avoiding vigorous foaming of the solution. Using an 8-channel pipette, add 50 μl of a diluted solution of CA-62-Acridinium conjugate to all wells (the total concentration of the conjugate in the wells will be X/2 μg/ml). Mix the reaction mixture thoroughly by gradually tapping the different sides of the microplate.
7. Close the microplate with a protective film and aluminium foil and incubate at room temperature from 20° C. to 25° C. for 2 hours.
8. Drain or aspirate the contents of the wells. Then, wash the microplate 6 times with distilled water using a plate washer or a wash bottle, by aspirating and immediate filling of the wells of each strip. Add 270 μl of liquid to each well during each wash cycle. At the end of the washing process, remove excess liquid by flipping the microplate on the absorbent paper or medical cloth.

### Stage 3. Detection Results

1. To detect the experiment data, set the “empty” filter (“<--->”, “blank”) in the luminometer.

If required, the “CA-62” Positive Control can be used, which does not require prior dilution and is introduced into the well of the microplate in 50 μl volume, followed by the addition of a dilute solution of the CA-62-Acridine conjugate.
To conduct an experiment for luminometers with ONE injector.

Using a dispenser, add 50 μl to each well of the microplate pre-activator solution (aqueous solution of 1 mmol/l HNO₃ and 0.1% H₂O₂, store in a dark place, protected from light) and put the microplate in the device. The luminometer injector should dispense 50 μl per well of the activator (1N NaOH) of the luminescent signal and read the results from each well from 0.25 to 1 second. The integration time must be set in accordance with the sensitivity of the luminometer. For the Anthos Lucy 2 luminometer, the recommended reading time is 1 second.
To conduct an experiment for luminometers with TWO injectors.

Set the dispensing volume of the injector 1 to 50 μl of the pre-activator solution (aqueous solution of 1 mmol/l HNO₃ and 0.1% H₂O₂) for each well, and the dispensing volume of the injector 2 to 50 μl 1N NaOH for each well. Waiting time between the dispensing of the injectors 1 and 2 should be set to 0.5 seconds. The reading of the results must be performed within 1 second after the end of the dispensing of the second injector. Read and record the relative light units (RLU) immediately.

### Stage 4. Calculation of the Marker for Epithelial Carcinomas Level

The calibration curve is a logarithmic function with a negative slope (the lower the unit of measurement, the larger the unit of CA-62). Using Microsoft Excel, calculate the values of CA-62 samples as follows:
1. Calculate the mean RLU value for each calibrator and each serum sample (in duplicates).
2. Plot a calibration curve: assign the measured RLU values of the calibrators along the y-axis, and measurements of the calibrators in CA-62 units along the x-axis. Change the format of the horizontal axis to a logarithmic scale by pressing the right button on the mouse. Determine the logarithmic function of the corresponding calibration curve. The coefficient of determination R² value should not be less than 0.9. Otherwise, it will be necessary to repeat the measurements for the entire series of samples.
3. To interpolate the measurement units of the CA-62 marker in U/ml, it is necessary to use the coefficients of the logarithmic equation y=a·Ln(x)+b, in which a and b are coefficients in the formula calculated by Excel (Table 1). Use the measured “y” values to solve the equation and determine the x value for each sample using the following equation: =EXP((RLU−b)/a). It must be remembered that the coefficient “a” will be a negative number. In this case, the calibration curve will be described by a logarithmic equation: y=−10.9 ln(x)+110.07, where a=−10.9 and b=110.07.

TABLE 1
Experimental data for constructing a calibration curve.

| | [CA-62], U/ml | | | | | | | |
|---|---|---|---|---|---|---|---|---|
| | 50 | 250 | 650 | 1250 | 2500 | 5000 | 7000 | 10000 |
| RLU | 70 | 50 | 35 | 30 | 22 | 18 | 15 | 9.8 |

4. Samples with RLU values that fall outside the scale of the standard calibration curve should not be extrapolated to this curve. Instead, the measurement results must be specified as greater than 10,000 U/ml or less than 50 U/ml, which represents the values of the largest and lowest CA-62 calibration standard, respectively.
5. Samples with CA-62 units greater than the largest calibrator can be diluted accordingly. The concentration of CA-62 should be measured again, after which the exact concentration can be determined by multiplying by the dilution factor.

Some parameters that were used in the test development:

Sensitivity is the percentage of those samples, for which the test correctly diagnosed the disease (as determined by the gold standard) as compared to the total number of samples with disease. Correctly identified positive samples from patients with cancer are called True Positives (TP). Those samples from cancer patients that were missed during testing are called False Negatives (FN). Thus, the sensitivity is described by the following equation: Sensitivity=TP/(TP+FN).

Test specificity is the proportion of samples that are tested negatively, which can be expressed by the equation: Specificity=TN/(TN+FP), where TN=True Negative samples (healthy individuals defined as negative for CA-62) and FP=False Positive samples (healthy individuals, incorrectly defined by the test as positives).

Characteristic ROC curve (Receiver Operating Characteristic): The ROC curve indicates the ratio of the test's sensitivity and its specificity (in fact, the inverse of specificity (1—Specificity) to obtain a curve, rather than a linear relationship). It indicates the ability of the test to distinguish between cancer patients and healthy individuals. A test, where there is no difference between groups of samples will show a diagonal between the axes of the coordinates. The more the difference between healthy controls and the samples with disease is observed, the more the curve will bend approaching the axes of coordinates. The ideal test (100% sensitivity and 100% specificity) will have a shape in the form of a right angle along the axes of coordinates.

### Stage 5. Interpretation of Results

Data analysis for patients with breast, prostate and ovarian carcinomas showed that a significant increase in CA-62 marker's level in the blood of patients as compared to the normal level of this marker in the serum of healthy individuals is directly associated to cancer development The cut-off value for CA-62 marker is 5000 U/ml, while the reference range, reflecting the lower and upper limit of the laboratory indicators for CA-62, ranges from 50 to 4999 U/ml. Thus, the results that go beyond the reference marker values and exceed a cut-off value of 5000 U/ml are sufficient for searching the reason for such expression of CA-62 cancer antigen, which can inform of a possible pathological process.

The sensitivity of determining the CA-62 cancer antigen in the blood of patients with early stages of cancer is from 80 to 95% with 0 to 95% specificity. This indicates the high potential of its use to clarify the oncological diagnosis and the extremely low risk of false positive results.

Normalization of CA-62 level after the effective antitumor treatment such as chemotherapy, radiotherapy, and surgery, as well as stabilisation of its normal values in the blood indicates the remission achieved in treated patients. An increased level of CA-62 marker during remission should be a signal for more in-depth study of the patients. A gradual increase of theca-62 level in the patients' serum may be a significant prognostic factor, indicating a negative dynamic effect or the recurrence of a pathological process.

Thus, an increased level of CA-62 cancer antigen in patient's blood alone or in a combination with other tumor markers has consistent indication on the presence of a pathological process and can be used appropriately for early cancer diagnosis and for detection of its aggressiveness.

### Test Limitations

The presence of citrate, oxalate, ethylenediaminetetraacetic acid (EDTA) or other chelating agents as well as heparin in the samples can cause incorrect test results. The correct test results can be obtained only in serum from venous blood. Blood plasma samples are not suitable for this test. Pregnant and lactating women should not be tested using this assay since these conditions are associated with an increased level of AFP, which affects the measurements of CA-62 glycoprotein in the blood. The presence of an acute inflammatory process can theoretically lead to a false positive result, and therefore it is required to avoid testing these patients before they are cured. An initial testing of patients for the marker for epithelial carcinomas CA-62 is recommended prior to the start of chemotherapy, as it may affect the measurements. During chemotherapy, it is possible to measure the CA-62 marker only under the supervision of a chemotherapist in the monitoring mode of cancer treatment using the dynamics of the marker for epithelial carcinomas. Previous studies have shown that about 5%-10% cancer patients' samples can be negative, depending on the type of cancer, its prevalence, stage of cancer and the differentiation of tumor cells. Also, from 2 to 5% of healthy people and up to ˜10% of patients with benign tumors can be tested positive (at this stage of research it is not known whether there are individual benign tumors and neoplasms in the process of transition to malignant neoplasms). In connection with the above, this CLIA test is designed to diagnose cancer at the early stages, to use for monitoring cancer treatment response and for disease development, their differentiation from benign tumors in the body, in order to further determine their localisation using modern diagnostic methods.

Technical task: Development of the diagnostic cancer test that is aimed to identify the early stages of cancer.

The technical result of the proposed invention is the design of a set of reagents for the early diagnosis of cancer, detection of tumor recurrence and monitoring cancer treatment response of human epithelial tumors by immunochemical analysis that has the following advantages over similar solutions:
1. The set of reagents allows identification of the early stages of epithelial tumors of the mammary gland, prostate gland and ovarian carcinomas, including cancer in situ, with a level of accuracy not achievable by other methods of in vitro diagnostics.
2. The set of regents has high sensitivity and specificity for the detection of primary malignant tumors (stages I-IV) compared to other tests that are based on tumor markers.
3. The set of reagents is highly effective for cancer monitoring chemotherapy for patients with lung cancer, stomach cancer, and colorectal cancer.
4. The set of reagents is highly sensitive in detecting tumor recurrences in patients with ovarian cancer and stomach cancer.

This technical result is achieved:

According to point 1—using unique monoclonal antibodies directed against the N-glycosidic epitope of the CA-62 glycoprotein and developed by hybridoma technology method. Monoclonal antibodies allow recognition of all forms of glycosides on the surface of the malignant cells with high accuracy. It helps to identify oncogenesis at the very beginning.

According to point 2—using a reliable one-step approach for CLIA with labelled CA-62 cancer antigen and highly sensitive flash Luminometer allows significantly increase the sensitivity and specificity of the developed set of reagents.

According to point 3—studying the dynamics of the marker for epithelial carcinomas during chemotherapy of patients with common forms of epithelial cancer is highly sensitive and can be used for monitoring cancer treatment response.

According to point 4—studying the dynamics of CA-62 marker for epithelial carcinomas during monitoring cancer treatment of patients with epithelial ovarian cancer after surgery is highly sensitive and can be used to detect cancer recurrence.

### EXAMPLES

### Example 1. The Investigation of a Blind Set of Samples 1 “Working Set of Samples with Breast Cancer, Benign Breast Diseases, and Healthy Controls”

TABLE 2
Characteristics of the Working panel of samples

| | Test | | | | | | | | | | | |
|---|---|---|---|---|---|---|---|---|---|---|---|---|
| | Breast | | Breast | | Breast | | Breast | | Benign | | | |
| | Cancer, | | Cancer, | | Cancer, | | Cancer, | | Breast | | Healthy | |
| | Stage 1 | | Stage 2 | | Stage 3 | | Stage 4 | | Disease | | Controls | |
| | N = 120 | | N = 180 | | N = 100 | | N = 100 | | N = 100 | | N = 120 | |
| CA-125, x̅ | 15 | U/ml | 22 | U/ml | 54 | U/ml | 82 | U/ml | 45 | U/ml | 12 | U/ml |
| Sensitivity | 41% | | 42% | | 62% | | 82% | | 44% | | 41% | |
| Specificity | 95% | | 84% | | 86% | | 88% | | 85% | | 95% | |
| CEA, x | 9 | ng/ml | 18 | ng/ml | 22 | ng/ml | 38 | ng/ml | 22 | ng/ml | 2.5 | ng/ml |
| Sensitivity | 39% | | 41% | | 60% | | 64% | | 34% | | 60% | |
| Specificity | 95% | | 83% | | 88% | | 87% | | 85% | | 88% | |
| CA-62, x̅ | 10550 | U/ml | 8500 | U/ml | 7520 | U/ml | 6700 | U/m | 4680 | U/ml | 3500 | U/ml |

| | | | | | | |
|---|---|---|---|---|---|---|
| Sensitivity | 94% | 97% | 93% | 89% | 72% | 100% |
| Specificity | 95% | 98% | 95% | 100% | 85% | 95% |

### Detection of the Early Stages (I and II) of Breast Cancer Using CA-62 Marker in a Set of Samples 1 of the “Working Set of Samples of Breast Cancer, Benign Breast Diseases, and Healthy Controls.”

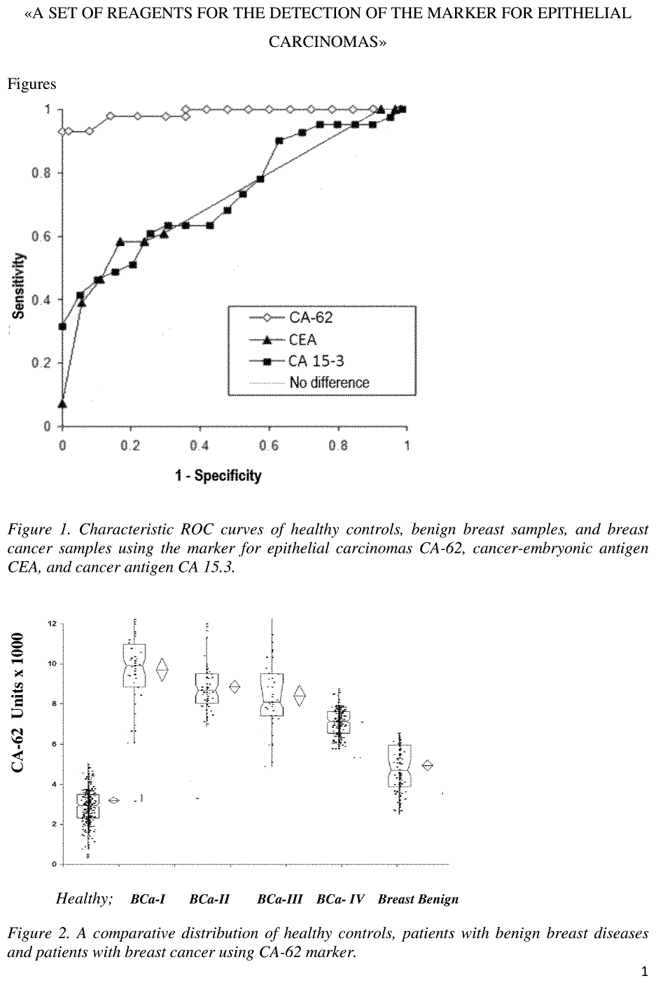

The p-value of the independent t-test corresponded to 4.12×10⁻²² when comparing healthy samples with samples of patients with breast cancer. When comparing serum samples of patients with benign and malignant tumors, the p-value for the t-test was detected to be 1.2×10⁻⁴. FIG. 1 shows the ROC curves for the three markers (CEA, CA-15-3, CA-62) by comparing the healthy control samples with samples of patients with breast adenocarcinoma. The area under the curve (AUC) for the CA-62 marker was 0.985. When using a cut-off value of 5000 U/ml for the CA-62 epithelial carcinoma marker, the sensitivity of the method was 94% with 95% of specificity. For the same samples with a 95% specificity, the sensitivity of the CEA test corresponded to only 39% (AUC: 0.723), and the sensitivity of the CA 15-3 test was 41% (AUC: 0.739). FIG. 2 demonstrated the distribution of control breast cancer samples, healthy controls, and samples with benign diseases using the CA-62 marker.

With an increase in the cut-off value to 7500 U/ml, a 100% specificity of the CA-62 test is achieved with the detection of 75% of the earliest stages of breast cancer. While maintaining 100% specificity, the sensitivity of the CEA and CA 15-3 tests was 32% and 37%, respectively. When comparing benign tumor samples with a panel of samples with breast cancer, a higher cut-off value is contributed to a better differentiation of these panels. At a cut-off of 7300 U/ml of CA-62, the sensitivity of the test was 72% with 85% specificity (AUC: 0.773). For the same samples with 85% specificity, the sensitivity of the CEA and CA 15-3 tests was 34% (AUC: 0.626) and 44% (AUC: 0.685), respectively.

### Detection of the Stages III and IV of Breast Cancer in a Set of Samples 1 of the “Working Set of Breast Cancer Samples, Benign Breast Diseases, and Healthy Controls.”

The ROC curve of CA-62 epithelial carcinoma marker, comparing control and benign samples with samples from patients with breast adenocarcinoma, has a value of AUC: 0.99. At a cut-off of 5000 U/ml of CA-62, the sensitivity of the test was 97% with a specificity of 95%. When comparing the serum of patients with benign and malignant breast tumors using a cut-off value of 5500 U/ml of CA-62 biomarker, the sensitivity of the test was 88% with 97% specificity (AUC: 0.97). When analysing the ROC curve using 4700 U/ml of CA-62, the sensitivity of the test was 89% with 100% specificity. The AUC value is 0.98.

When comparing serum samples of patients with benign breast samples and breast cancer samples, raising the cut-off value of the CA-62 tumor marker to 7300 U/ml allowed the differentiation of categories of patients much better, while the sensitivity of the CA-62 test was 74% with 85% specificity (AUC: 0.783). The same samples with 85% specificity showed 34% sensitivity of the CEA test (AUC: 0.626) and 44% sensitivity of the CA 15-3 test (AUC: 0.685).

### Conclusions:

1. Marker for epithelial carcinoma CA-62 can distinguish with high sensitivity and specificity healthy individuals and patients with benign breast diseases from patients with breast cancer.
2. The developed immunological test can detect early stages of breast cancer with a high level of sensitivity and specificity, which are not currently achievable by other tumor markers. Choosing a low CA-62 cut-off value (4700-5000 U/ml) increases the probability of detecting oncology in patients: sensitivity from 93% to 94% with 95% specificity when compared to healthy patients.
3. In this study, the diagnostic value of the assay using the CA-62 cancer antigen have better results for the tested samples than using the CEA and CA 15-3 tests (sensitivity 39% and 41%, respectively, for both markers with 95% specificity).

### Example 2. The Investigation of the Set of Samples 2 “Working Set of Samples with Ovarian Cancer, Benign Gynecological Diseases, and Healthy Controls”

### Samples:

Serum samples from 200 patients with a confirmed diagnosis of ovarian carcinoma (stage I/II=83 and stage III/IV=117), 50 samples of patients with benign diseases, and 105 healthy control samples were included in the study. From the 200 patients with histologically confirmed malignant tumors, 60% were detected with serous carcinomas, 20% with mucinous carcinomas, 10% with endometriotic carcinomas, and 10% with undifferentiated carcinomas. All serum samples were collected from patients before treatment. The diagnoses were confirmed by histological examination prior to the start of this study. Samples were taken into sterile containers with a blood clot activator. After the coagulation of the blood, the samples were centrifuged and frozen at −30° C.

### Study Design:

The serum level of the CA-62 epithelial carcinoma marker was measured using chemiluminescence immunoassay (CLIA). The characteristics of the samples of the working panel 2 are presented in Table 3.

TABLE 3
Characteristics the Working Set 2 samples “Working
set of samples of ovarian cancer, benign gynecological
diseases, and healthy controls.”

| | | | | | Mean |
|---|---|---|---|---|---|
| Biomarker | Samples | Quantity | 95% | Min/Max | value |
| Cancer | Healthy | 105 | 4400 | 50/4800 | 3500 |
| antigen | Epithelial | 200 | 12621 | 2917/17422 | 7922 |
| CA-62 | cancer, all | | | | |
| U/ml | stages | | | | |
| | Carcinoma, | 83 | 10043 | 4946/17422 | 9217 |
| | stages I, II | | | | |
| | Carcinoma, | 117 | 12796 | 2917/14214 | 7624 |
| | stages III, IV | | | | |
| | Benign | 50 | CA-62 | 2500/3850 | 3200 |
| | tumors | | <4000 | | |
| | | | U/ml | | |
| Cancer | Healthy | 105 | 28 | 0/35 | 10.2 |
| antigen | Epithelial | 200 | 1695 | 0/1898 | 585 |
| CA-125 | cancer, all | | | | |
| U/ml | stages | | | | |
| | Carcinoma, | 83 | 672 | 0/1028 | 273 |
| | stages I, II | | | | |
| | Carcinoma, | 117 | 1782 | 0/1898 | 678 |
| | stages III, IV | | | | |
| | Benign | 50 | CA-125 | 20/120 | 80 |
| | tumors | | >35 | | |
| | | | ng/ml | | |

### The Results Obtained:

Table 3 shows the descriptive statistics of CA-62 and CA-125 cancer antigens in the serum of healthy controls and a working panel of patients with different stages of ovarian cancer. Linear regression analysis demonstrates a low correlation coefficient between the values of the CA-62 marker and the CA-125 cancer antigen in all samples (r²=0.1318 [r=0.36]ϰr²=0.0056 [r=0.07] in samples containing only cancer patients). The lack of correlation between these markers means that their combination may improve their individual diagnostic abilities in cancer detection.

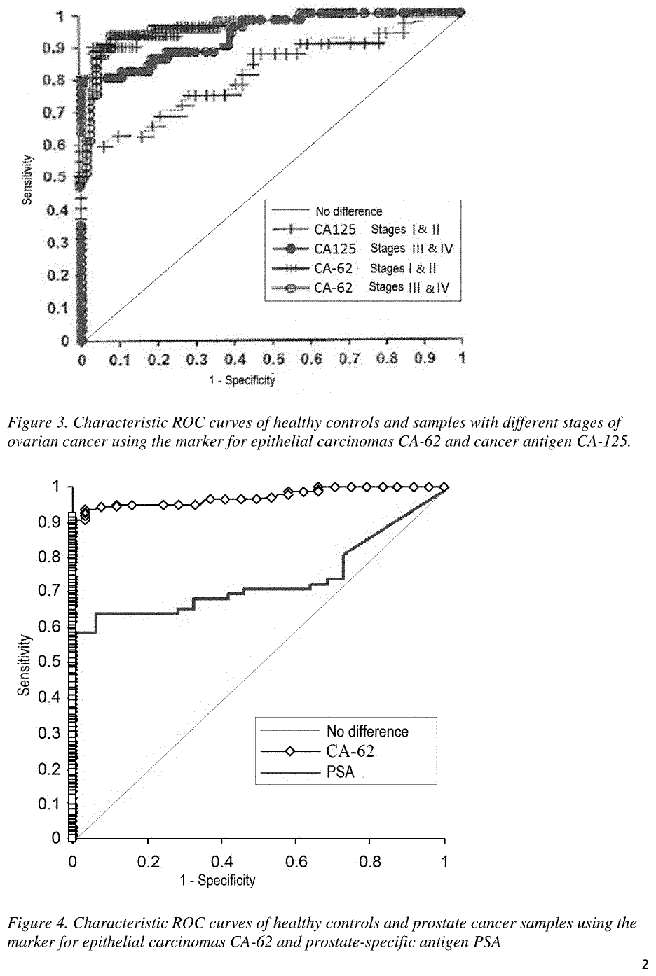

FIG. 3 shows the ROC curves of CA-62 and CA-125 cancer antigens for all stages of ovarian cancer. The ability to differentiate oncology patients' samples from the healthy control samples is significantly higher in the CA-62 biomarker, especially in the early stages of cancer, where the diagnostic value of the CA-125 biomarker is lower (the AUC value for CA-62 was 0.96 compared to 0.805 for CA-125).

It is important to note, that while the sensitivity of the CA-125 cancer antigen is significantly lower in the early stages of cancer compared with the later stages, the sensitivity of the CA-62 marker for epithelial carcinomas remains high at all stages of the disease.

A detailed analysis of various malignant ovarian tumors using the CA-62 epithelial carcinoma marker and the CA-125 cancer antigen is shown in Table 4:

TABLE 4
Characteristic features of the studied samples of ovarian cancer.

| | Characteristic | Characteristic | |
|---|---|---|---|
| | interval of CA- | interval of CA- | |
| Type of disease | 62, U/ml | 125, ng/ml | 95% samples |
| Ovarian | 4900-15000 U/ml | 8-400+ ng/ml | CA-62 = |
| adeno- | 67%: 6000-10000 | 23%: <35 | 15000 U/ml |
| carcinoma | 23%: >10000 | 77%: >35, [symbol] | CA-125 = |
| | 10%: <6000 | 44%: >400 ng/ml | 500 ng/ml |
| | Mean value | Mean value | |
| | CA-62 = | CA-125 = | |
| | 7578 U/ml | 225 ng/ml | |
| Endometrioid | 6500-9000 U/ml | 20-400 ng/ml | CA-62 = |
| ovarian | Mean value | Mean value | 9000 U/ml |
| adeno- | CA-62 = | CA-125 = | CA-125 = |
| carcinoma | 7750 U/ml | 210 ng/ml | 250 ng/ml |
| Ovarian serous | 6500-20000 U/ml | 0-1250 ng/ml | CA-62 = |
| cyst adeno- | 85%: >8000-28000 | 33%: <35 ng/ml | 28000 U/ml |
| carcinoma | 22%: >15000 | 67%: >35 ng/ml, | CA-125 = |
| | 15%: <7000 | [symbol] | 220 ng/ml |
| | Mean value | 55%: >100 ng/ml | |
| | CA-62 = | Mean value | |
| | 10230 U/ml | CA-125 = | |
| | | 175 ng/ml | |
| Ovarian | 8000-17000 U/ml | 0-32 ng/ml | CA-62 = |
| papillary | 40%: >8000 | 100%: <35 ng/ml | 12000 U/ml |
| carcinoma | 60%: >10000 | Mean value | CA-125 = |
| | Mean value | CA-125 = | <32 ng/ml |
| | CA-62 = | 19 ng/ml | |
| | 10740 U/ml | | |
| Mucinous | 5500-7000 U/ml | 120 ng/ml | CA-62 = |
| ovarian | Mean value | Mean value | 6500 U/ml |
| carcinoma | CA-62 = | CA-125 = | CA-125 = |
| | 6000 U/ml | 473 ng/ml | 500 ng/ml |
| Andro- | 5000-6000 U/ml | 1000 ng/ml | CA-62 = |
| blastoma, | Mean Value | Mean value | 5000-6000 |
| Sertoli-Leydig | CA-62 = | CA-125 = | U/ml |
| cell tumors | 5500 U/ml | 1200 ng/ml | CA-125 = |
| | | | >1000 ng/ml |
| Malignant | 6000-8000 U/ml | 250 ng/ml | CA-62 = |
| metastatic | Mean value | Mean value | 8000 U/ml |
| Krukenberg | CA-62 = | CA-125 = | CA-125 = |
| tumor | 7000 U/ml | 325 ng/ml | >250 ng/ml |
| Benign | <4000 U/ml | 35 ng/ml | CA-62 = |
| ovarian | | | <4000 U/ml |
| disease | | | CA-125 = |
| | | | >35 ng/ml |

Table 4 shows no correlation between the CA-62 and CA-125 biomarkers in the tested samples. No correlation gives a possibility to use both of these markers in a coordinated manner to increase sensitivity and tissue-specificity in the identification of the earliest stages of ovarian carcinoma.

### Detection of Early Stages of Ovarian Cancer Using the CA-62 Marker in the Set of Samples 2 of the “Working Set of Ovarian Cancer Samples, Samples of Benign Gynecological Diseases, and Healthy Controls.”

The conducted study showed that the sensitivity of the CA-62 test is significantly higher when determining the earliest stages of ovarian cancer compared to the CA-125 marker. Using a cut-off value of 5000 U/ml for CA-62 cancer antigen, the test with 95% specificity showed 95% sensitivity for the detection of stage I samples, 90% sensitivity for stage II, and 87% for later stages (III and IV) of ovarian cancer.

Unlike the CA-62 biomarker, the sensitivity of the CA-125 test is significantly increased in the later stages of ovarian cancer. While for the CA-62 test, the sensitivity remains high at all stages of the disease. The CA-125 test has a good prognostic ability in monitoring the treatment of ovarian cancer, diagnosing its metastasis and evaluating the effectiveness of therapy. A significant increase in the CA-125 marker clearly indicates the occurrence of a relapse of the disease. A comparative analysis of the diagnostic significance of CA-62 and CA-125 tests is presented in Table 5:

TABLE 5
The diagnostic value of CA-62 and CA-125 tests

| | | | | Sensitivity % | | |
|---|---|---|---|---|---|---|
| | | | | Stage | Stage | Stage |
| Test | Cut-off value | | Specificity | I | II | III&IV |
| CLIA CA-62 | 5000 | U/ml | 95% | 92% | 90% | 87% |
| ELISA CA-125 | 35 | ng/ml | 85% | 25% | 55% | 80% |

The results of the comparative analysis show that the use of serum values of the CA-62 marker makes it quite feasible to obtain a screening test for ovarian carcinoma. This test will be able to detect the early stages of ovarian cancer, at which the cure rate is at maximum.

### Conclusions:

1. A comparative analysis of the CA-62 and CA-125 biomarkers on the Control Set of samples 2 showed that the sensitivity and specificity of the CA-62 epithelial carcinoma marker significantly exceed these characteristics for CA-125 for all stages of ovarian cancer.
2. The serum level of the CA-62 biomarker provides the high specificity and sensitivity required for screening for ovarian cancer, and in particular for detecting the early stages of this disease, when the cure rate is the highest.
3. A selection of high cut-off value will exclude healthy people. In this case, any patients tested positively is very likely to have some kind of pathology that will require its diagnosis, and in some cases, its treatment. In these cases, the test results should be considered as beneficial for the patient, regardless of the specific pathology.

This study provides a framework for the development of a strategy that can provide an economical and sensitive approach for detection the early stages of ovarian cancer. Therefore, it will also help to reduce mortality caused by this disease.

### Example 3. The Investigation of the Set of Samples 3 “Working Set of Samples with Prostate Cancer, Benign Prostate Diseases, and Healthy Controls”

### Samples:

A set of serum samples from patients with early and advanced stages of the prostate cancer and patients with benign prostate diseases, in particular with prostatic hyperplasia, was examined.

### Objectives of the Study:

Comparative analysis of the marker for epithelial carcinomas CA-62 with prostate-specific antigen (PSA) as an alternative method for the early detection of the prostate cancer.
Studying the possibility of using two cancer markers (PSA and CA-62) in comparison with the combination (free PSA/total PSA) to increase sensitivity in detecting the earliest stages of prostate cancer and to increase the survival rate.
Exploring the possibility of using a combination of two markers (PSA and CA-62) to reduce the number of unnecessary biopsies for patients with benign prostatic hyperplasia (BPH).

### Study Design:

All blood samples were collected and processed according to standard procedures using vacuum containers with a clotting activator and a separating gel. Then all samples were centrifuged. The serum was then separated and frozen for storage at −20° C. Before use, all samples were aliquoted and treated with heat at 56° C. for 30 minutes.

To analyze the obtained data, we used a one-step competent serum immunoassay on monoclonal antibodies developed by us, specific to the N-glycoprotein CA-62. The “CA-62” serum test was developed as a single-stage “competitive” variant of solid-phase chemiluminescence immunoassay (CLIA) using highly specific anti-CA-62 antibodies. The circulating level of total and free PSA was measured using the AxSYM® system (Abbott Labs, USA).

After receiving the measurement results and the disclosure of the diagnosis for the studied samples, all samples were divided into 3 groups: healthy controls (24), samples with benign prostate diseases (118) and a group of samples with prostate cancer (212). The panel contains 212 samples with histologically confirmed prostate cancer stages: Stage I—22 samples, Stage II—30 samples, Stage III—70 samples and Stage IV—90 samples and 118 samples with histologically confirmed prostatic hyperplasia (BPH). All malignant and benign samples were collected from patients before treatment, and all diagnoses were confirmed prior to the study using histological examination.

### The Obtained Results:

The results are presented in Table 6:

TABLE 6
Descriptive statistics of CA-62, free PSA/total PSA serum level
in prostate cancer samples and prostate hyperplasia (BPH) samples

| | | | |
|---|---|---|---|
| | Mean | Standard | Median |
| Marker | value | deviation | value |

All samples with prostate cancer

| | | | |
|---|---|---|---|
| PSA ng/ml | 261 | 1118 | 6.63 |
| Free PSA/total PSA | 0.15 | 0.13 | 0.13 |
| CA-62 U/ml | 9479 | 6,139 | 7933 |

All samples with Benign Prostate Hyperplasia (BPH)

| | | | |
|---|---|---|---|
| PSA ng/ml | 6.92 | 6.27 | 6.005 |
| Free PSA/total PSA | 0.20 | 0.13 | 0.17 |
| CA-62 U/ml | 4315 | 4,061 | 2823 |

Samples with prostate cancer with PSA level of 2-20 ng/ml

| | | | |
|---|---|---|---|
| PSA ng/ml | 7.98 | 4.04 | 7.36 |
| Free PSA/total PSA | 0.15 | 0.08 | 0.15 |
| CA-62 U/ml | 8828 | 4,765 | 7676 |

Samples with Benign Prostate Hyperplasia (BPH) with
PSA level of 2-20 ng/ml

| | | | |
|---|---|---|---|
| PSA ng/ml | 7.27 | 3.60 | 6.605 |
| Free PSA/total PSA | 0.18 | 0.08 | 0.16 |
| CA-62 U/ml | 4484 | 4343 | 2753 |

Patients with PSA interval from 2 to 20 ng/ml: 131 of 212 oncological patients (62%) and 98 of 118 patients with prostate hyperplasia (83%) have from 2 to 20 ng/ml PSA (Table 7).

TABLE 7
Sensitivity and specificity of combination of markers
in samples with PSA level from 2 to 20 ng/ml

| Combination of markers | Sensitivity | Specificity |
|---|---|---|
| Free PSA/total PSA in samples with PSA | 122/131 (93%) | 11/98 (11%) |
| of 2-20 ng/ml | | |
| CA-62 in samples with PSA of 2-20 ng/ml | 120/131 (93%) | 64/98 (65%) |

Detection of Early Stages of Prostate Cancer with CA-62 Marker in the Set of Samples 3 of the “Working Set of Prostate Cancer Samples, Benign Prostate Disease Samples, and Healthy Controls”.

An important task of the study was to conduct a comparative analysis of the CA-62 marker of epithelial carcinomas with the prostate-specific antigen (PSA) in their ability to detect the early stages (I and II) of prostate cancer. Of the 52 samples corresponding to stages I and II, the CA-62 test revealed 42 out of 52 (81%) positive sample, while the PSA test was positive in only 15 out of 52 (29%) cases. FIG. 4 shows a comparison of the characteristic curves for the studied working panel of prostate cancer samples using CA-62 and PSA serum markers.

### Conclusions:

The study led to the following conclusions:
1. A serum test with the marker for epithelial carcinoma CA-62 with a cut-off value of 3.900 U/ml helps to identify 93% of the early prostate cancer stages. It is performed on samples with a PSA level of 2-20 ng/ml or 4-10 ng/ml. It also helps to reduce the number of unnecessary biopsies significantly.
2. Combinational use of the PSA and serum test with the CA-62 epithelial carcinoma marker to distinguish benign prostatic hyperplasia (BPH) from prostate cancer increases the specificity of the test 4-6 times and reduces the need for invasive intervention.
3. The ratio of total PSA and free PSA obtained for the studied samples is in accordance with the literature data, which confirms that these samples can represent a large population. This validation is very important because it allows to summarise the results and conclude that the use of a specific CA-62 epithelial carcinoma marker can significantly reduce the number of complications and reduce the risk of death associated with prostate biopsy.

The research results are prepared for publication.

### Example 4. The Investigation of the Chemotherapy Effectiveness and Detection of Relapses in Patients with Metastatic Epithelial Tumors Using the New CA-62 Epithelial Carcinoma Marker

### Purpose of the Study:

1. Assessment of the dynamic's effectiveness of the CA-62 biomarker level for monitoring chemotherapy in patients with various solid tumors.
2. Detection of the correlations between the expression level of the CA-62 marker and the tumor response to treatment in accordance with the RECIST criterion.
3. Detection of disease recurrence with the help of epithelial carcinoma marker.

### Study Design:

The study was conducted on 100 patients with various solid tumors (stomach cancer, single cell lung cancer, colorectal cancer, and endocrine cancer) including stabilised patients (36%) who had previously received systemic treatment and are in remission. Blood from patients was collected and processed before, during, and at the end of the treatment. The blood sampling was held at the N.N. Blokhin Russian Cancer Research Centre (Moscow, Russia) in accordance with approved standard protocols. The total duration of the observation was 12 months. An objective response was evaluated according to computed tomography of the chest, abdominal organs, pelvic organs, ultrasound, brain MRI and bone scintigraphy every 2-3 chemotherapy courses, according to the criteria of RECIST 1.1. The correlation was assessed between the CA-62 marker and the course of the disease. This determines the relationship of the disease progression according to RECIST 1.1 with an increase in the CA-62 value over 4600 U/ml, and positive dynamics or stabilisation of the disease—with its decrease.

### Patients:

The study was conducted from August 2015 to December 2016. The coded clinical study included patients with locally-advanced (stages II and III) and metastatic forms of cancer, confirmed morphological results with at least one measurable primary tumor according to RECIST criteria, general health at the time of inclusion by ECOG from 0 to 2 and satisfactory laboratory values. Measurement of the CA-62 level in the serum of each patient was carried out once a month during chemotherapy (2-5 months) and continued in the same mode after treatment (3 months).

Blood samples of 120 patients with confirmed diagnoses (stages IIb=22 and stage III/IV=88), as well as blood samples of 28 healthy people as controls, were included in the study. Twenty patients who did not undergo regular examinations necessary for solving research problems were excluded from the final analysis. The final analysis of the study also included 100 patients who suffered from various solid tumors: 34—stomach cancer, 10—colorectal cancer (CRC), 10—ovarian cancer, 22—small cell lung cancer (SCLC), 7—neuroendocrine tumors of the gastrointestinal tract and pancreatic cancer, 4—cervical cancer, 3—pleural mesothelioma, 4—breast cancer, 4—adrenocortical cancer.

### The Obtained Results:

Detection of tumor recurrences: measuring the CA-62 level in the serum of each patient during chemotherapy (3-6 months) revealed recurrences in 8% of patients before their instrumental confirmation. The results of the CA-62 test for detecting recurrences in patients with advanced epithelial tumors are presented in Table 8.

TABLE 8
The diagnostic value of CA-62 test in
the detection of tumor recurrence

| | TRUE RESULT | | |
|---|---|---|---|
| RESULT OF | Relapse | No relapse | |
| CA-62 TEST | (True positive) | (True negative) | TOTAL |
| Positive | 8/8 (100%) | 0/0 (100%) | 8 |
| Negative | 0/0 (100%) | 92/92 (100%) | 92 |
| Total | 8 | 92 | 100 |

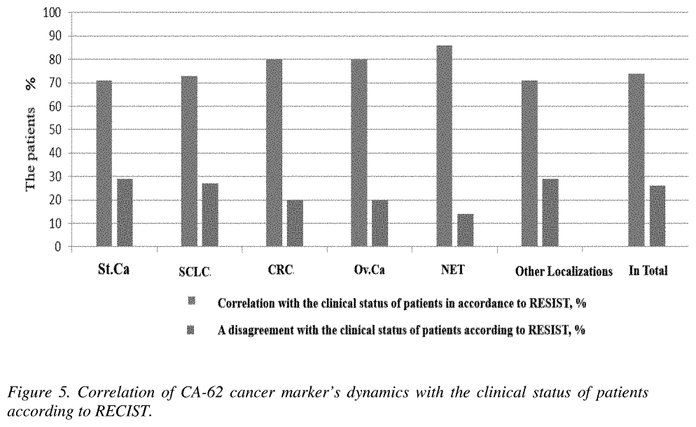

Monitoring chemotherapy in patients with locally advanced and metastatic cancers: before treatment, an increased CA-62 biomarker level was observed in 76% of patients. The decrease to normal reference values (50-4600 U/ml) was detected in 56% of patients after chemotherapy, while in 20% of patients it remained elevated. Moreover, in all 20% of patients with a consistently high level of the CA-62 biomarker expression, regardless of the treatment, disease progression was observed. In 36% of patients, no changes in CA-62 expression were observed during chemotherapy. After the start of treatment, the progression of the disease was observed in 22% of this group, and stabilization was achieved in 14% of patients. FIG. 5 shows the correlation of the CA-62 marker level with the tumor response to treatment in accordance with the RECIST criteria. Table 9 summarizes the data for all patient groups.

TABLE 9
Summary of the coded clinical trial data for all groups of patients according to the RECIST criterion

| | | | | Clinical status | | Clinical status |
|---|---|---|---|---|---|---|
| | | | | of patients | | of patients |
| | Number of | | Coincidence | according to | Disagreement | according to |
| Localisation | patients | Treatment | with clinical | RECIST | with clinical | RECIST |
| Stomach | 34 | Platinum, | 71% | 44% of cases - | 29% | 26% of cases - |
| cancer | | Fluoropyrimidine, | | progression of the | | the progression |
| | | or Targeted | | disease with | | of the disease, |
| | | Therapy for | | metastases in the | | metastases in |
| | | HER2 | | peritoneum and in | | the peritoneum, |
| | | Mutations. | | the liver. | | in the liver, |
| | | | | 27% of cases - | | ascites. |
| | | | | stabilisation of | | |
| | | | | patients. | | |
| | | | | In some cases - the | | |
| | | | | full effect of | | |
| | | | | treatment. | | |
| Colorectal | 10 | Fluoropyrimidines, | 80% | 50% of cases - | 20% | 20% of cases - |
| cancer | | platinum | | progression of the | | progression of |
| | | derivatives, | | disease, metastases | | the disease, |
| | | anticancer drugs | | to the liver, lungs, | | metastases to |
| | | of plant origin | | peritoneum, ascites. | | the liver, lungs. |
| | | and targeted | | 30% of cases - | | |
| | | therapy for | | stabilisation process. | | |
| | | mutations of K- | | | | |
| | | RAS, N-RAS | | | | |
| | | and B-RAF | | | | |
| | | proteins. | | | | |
| Small cell | 22 | Platinum | 73% | 46% of cases - | 27% | 27% of cases - |
| lung cancer | | derivatives, | | progression of the | | progression of |
| | | topoisomerase | | disease, metastases | | the disease in |
| | | inhibitors and | | in mediastinal | | all patients, |
| | | anticancer drugs | | lymph nodes, brain. | | metastases in |
| | | of plant origin, | | 27% of cases - | | the lymph |
| | | radiation | | stabilisation process. | | nodes of the |
| | | therapy to the | | | | root of the |
| | | primary tumor | | | | lung, |
| | | and the area of | | | | mediastinum, |
| | | the | | | | liver and |
| | | mediastinum, as | | | | bones. |
| | | well as brain | | | | |
| | | irradiation. | | | | |
| Ovarian | 10 | Platinum- | 80% | 70% of cases - | 20% | 20% of cases - |
| cancer | | containing | | progression of the | | progression of |
| | | drugs, taxanes | | disease, metastases | | the disease, |
| | | and anticancer | | in the peritoneum, | | metastases in |
| | | antibiotics. | | lymph nodes, the | | the peritoneum, |
| | | | | growth of residual | | pleura, |
| | | | | tumors in the pelvis. | | retroperitoneal |
| | | | | In some cases, | | lymph nodes, |
| | | | | metastases in the | | ascites. |
| | | | | bone, in the lower | | |
| | | | | mediastinum, | | |
| | | | | ascites. | | |
| | | | | 10% of cases - | | |
| | | | | stabilisation process | | |
| Neuroendocrine | 7 | Platinum and | 86% | 43% of cases - | 14% | 14% of cases - |
| tumors of | | fluoropyrimidine | | stabilisation of the | | progression of |
| the | | derivatives, | | process | | the disease |
| gastrointestinal | | biotherapy with | | 43% of cases - | | with |
| tract and | | somatostatin | | progression of the | | metastases in |
| pancreas | | analogues. | | disease with | | the peritoneum, |
| | | | | metastases in the | | ascites. |
| | | | | retroperitoneal and | | |
| | | | | intraperitoneal | | |
| | | | | lymph nodes. | | |
| Other | 17 | Alkylating | 71% | 47% of cases - | 29% | 29% of cases - |
| localisations | | agents, | | progression of the | | progression of |
| | | antimetabolites, | | disease. Depending | | the disease. |
| | | anticancer | | on the primary | | Depending on |
| | | antibiotics, | | tumor, metastases in | | the primary |
| | | taxanes and | | the peritoneum, | | tumor, |
| | | vinca alkaloids. | | liver, lungs, pleura. | | metastases to |
| | | | | 24% of cases - | | the pelvic or |
| | | | | stabilisation of | | axillary lymph |
| | | | | process. | | nodes. |
| Summary | 100 | | 75% | | 25% | |

### Summary of the Obtained Results:

1. The recurrent diseases were recorded in 8% of patients, that also coincided with the dynamics of the CA-62 marker.
2. The dynamics of the changes in CA-62 marker in the course of chemotherapy in patients with stomach cancer, small-cell lung cancer (SCLC) and colorectal cancer (CRC) showed a good correlation (71-80%, depending on the localisation of tumors) with the general conditions of patients according to the RECIST criteria.
3. The possibility of using CA-62 biomarker for monitoring diseases and evaluating the performance of the systemic treatment is shown.

### Conclusions:

From the results obtained, out of 100 patients with malignant tumors of various localisations, the dynamic changes in the level of CA-62 biomarker during treatment were directly related to the tumor response to chemotherapy in 79% of patients. At the same time, a consistently high CA-62 marker level during multiple courses of chemotherapy may suggest its inefficiency and the possible changes to the treatment regimen. At the same time, a consistently low level of CA-62 marker may suggest a good response of the tumor to the treatment, and with its increase—the likelihood of progression.

Analysis of the obtained data allows assuming that the expression of the CA-62 marker correlates well with the tumor growth response to the treatment. The dynamics of the CA-62 marker can serve as an indicator of tumor inhibition or growth, evaluation of the performed treatment or detection of resistance to chemotherapy. Later, this approach can be successfully used in clinical practice for monitoring malignant tumors of the gastrointestinal tract, ovaries, lung, and neuroendocrine tumors.

### BRIEF DESCRIPTION OF THE FIGURES

### 1. FIG. 1

FIG. 1 demonstrates the ROC curves for the three markers (CEA, CA-15-3, CA-62) by comparing the healthy control samples with samples of patients with breast adenocarcinoma.

### 2. FIG. 2

FIG. 2 demonstrates the distribution of control breast cancer samples, healthy controls and samples with benign diseases using the CA-62 marker.

### 3. FIG. 3

FIG. 3 shows the ROC curves of CA-62 and CA-125 cancer antigens for all stages of ovarian cancer.

### 4. FIG. 4

FIG. 4 shows a comparison of the characteristic curves for the studied working panel of prostate cancer samples using CA-62 and PSA serum markers.

### 5. FIG. 5

FIG. 5 demonstrates the correlation of the CA-62 marker level with the tumor response to treatment in accordance with the RECIST criteria.

### REFERENCES

1. Battista R N, Grover S A. Early detection of Cancer: an overview. Annual Review of Public Health. 1988; 9:31-45.
2. Malati T. Tumor markers: An overview. Indian Journal of Clinical Biochemistry. 2007; 22(2):17-31.
3. Chigireva I B, Khasanov R S, Gilyazutdinov I A, Panchenko S V, Shakirov K T. Early diagnosis of prostate cancer. Practical Medicine. 2013; 108-11.
4. Humphrey P A. The role of tumor markers in the early detection of cancer. Seminars in Surgical Oncology. 1989; 5(3):186-93.
5. Meany D L, Sokoll L J, Chan D W. Early Detection of Cancer: Immunoassays for Plasma Tumor Markers. Expert Opinion on Medical Diagnostics. 2009; 3(6):597-605.
6. Marella S. Prognostic and Predictive Markers in Early Detection of Different Types of Cancers for Selected Organ Sites IOSR Journal of Pharmacy and Biological Sciences (IOSR-JPBS). 2013; 8(4):25-42.
7. Helfand B T, Conran C A, Xu J, Catalona W J. A multiparametric approach to improve upon existing prostate cancer screening and biopsy recommendations. Current Opinion in Urology. 2017; 27(5):475-80.
8. Dani H, Loeb S. The role of prostate cancer biomarkers in undiagnosed men. Current Opinion in Urology. 2017; 27(3):210-216.
9. Ankerst D P, Thompson I M. Sensitivity and specificity of prostate-specific antigen for prostate cancer detection with high rates of biopsy verification. Archivio Italiano di Urologia e Andrologia. 2006; 78(4):125-9.
10. Stavridis S, Saidi S, Lekovski L, Dohcev S, Spasovski G. Screening for prostate cancer: a controversy or fact. Hippokratia Journal. 2010; 14(3):170-5.
11. Kristiansen S, Jørgensen LM, Hansen M H, Nielsen D, Sölétormos G. Concordance of Hypermethylated DNA and the Tumor Markers CA 15-3, CEA, and TPA in Serum during Monitoring of Patients with Advanced Breast Cancer. BioMed Research International. 2015; 2015:986024.
12. Berghuis A M S, Koffijberg H, Prakash J, Terstappen L W, IJzerman MJ. Detecting blood-based biomarkers in metastatic breast cancer: a systematic review of their current status and clinical utility. International Journal of Molecular Sciences. 2017; 18(2):363.
13. Havrilesky L J, Whitehead C M, Rubatt J M, Cheek R L, Groelke J, He Q, Malinowski D P, Fischer T J, Berchuck A. Evaluation of biomarker panels for early stage ovarian cancer detection and monitoring for disease recurrence. Gynecologic Oncology. 2008; 110(3):374-382.
14. Sergeeva N. S., Marshutina N. V. Tumor-associated markers in screening programs aimed at active detection of ovarian cancer: reality, problems and prospects, Practical Oncology, 2010; 11(2):110-119.

1. A set of reagents for detecting the marker for epithelial carcinomas CA-62 in the blood serum of patients for early cancer diagnosis, detection of cancer recurrences and monitoring cancer treatment of human epithelial tumors by immunochemical analysis, which has higher sensitivity and specificity and is different from all currently available serum markers, and includes a microplate with immobilized antibodies to the cancer antigen CA-62, which are monoclonal antibodies of IgM class directed against the N-glycosylic epitope of the CA-62 glycoprotein, CA-62-Acridinium conjugate, standard calibrators CA-62 in the measurement range from 10 to 30,000 U/ml, including or not including CA-62 positive control, working buffer solution, pH from 2.0 to 8.0, containing Tween-80.
